(19) United States
(12) Patent Application Publication
(10) Pub. No.: US 2005/0063960 A1
(43) Pub. Date: Mar. 24, 2005

(54) Dna construct for assessing thymic function activity and therapeutical uses thereof

(76) Inventors: Jean-Francois Poulin, Quebec (CA); Remi Cheynier, Rambouillet (FR); Martin Bourbonniere, Montreal (CA); Rafick-Pierre Sekaly, Saint-Laurent (CA); Dominique Gauchat-Feiss, Outremont (CA)

Correspondence Address: David S Resnick;Nixon Peabody, 100 Summer Street, Boston, MA, 02110, US

(21) Appl. No.: 10/496,916

(22) PCT Filed: Nov. 29, 2002
(86) PCT No.: PCT/CA02/01839

Related U.S. Application Data
(60) Provisional application No. 60/333,764, filed on Nov. 29, 2001.

(51) Int. Cl. A61K 48/00; C12N 5/08

(52) U.S. Cl. 424/93.21; 435/372; 514/44; 536/23.5

(57) ABSTRACT

The present invention relates to a DNA construct for in vivo expression in an excision DNA circle created by DNA recombination machinery in T cells from a non-human mammal which comprises two recombination signal sequences (RSS) consensus sequences flanking a promoter, an enhancer and a reporter gene, wherein the excision DNA circle is diluted out after cellular division and the excision DNA circle is detected by expression of the reporter gene and the detection is indicative of thymic function activity of the mammal. The present invention also relates to a T cell transiently transfected with the DNA construct of the present invention, the cell expressing quantifiable levels of reporter gene for green fluorescent protein (GFP) for determining enhancing/decreasing thymic exportation, a mammal comprising the DNA construct of the present invention and methods thereof.

### BACKGROUND OF THE INVENTION

1. Field of the Invention

This invention relates to a DNA construct for assessing thymic function activity of a mammal, a DNA construct for screening drugs enhancing and/or decreasing thymic function of a mammal, and a method for detecting and/or isolating T cells recently emigrated from the thymus among others.

2. Description of Prior Art

The human thymus is responsible for the differentiation of immature thymocytes into mature T lymphocyte expressing either the CD4 or the CD8 molecule. During this process, thymocytes rearrange their genomic DNA at the T cell receptor (TCR) α and β loci to generate TCR molecules that will be further selected during positive/negative selection. TCR gene rearrangement, mediated by recombination activating genes (RAG) 1 and 2, leads to the generation of stable TCRα and β recombination circles (TRECs) that do not replicate and that are diluted out during subsequent cellular proliferation. Each type of gene rearrangement event (δRec→ψJα, Vα→Jα, Dβ→Jβ and Vβ→DβJβ) generates a unique TREC that will have a distinct primary nucleotide sequence. Using PCR-based assays, several groups have shown that it is possible to evaluate the frequency of TRECs in T cell populations. These extrachromosomal circular DNA molecules were shown to be at a very high frequency in FACS-purified CD4⁺CD45RA⁺CD62L⁺ (naïve) T cells (Poulin, J.-F. et al., J. Exp.Med., 1999) and are now considered surrogate markers of recent thymic emigrants (RTEs) (Douek, D. C. et al., Nature, 1998). TREC quantification has become a direct indicator of ongoing thymopoiesis (Haynes, B. F. et al., Ann. Rev. Immunol., 2000).

At this date, no exclusive cell surface molecule specific to the human, macaque or mouse RTE population has been identified. In the chicken, chT1, an Ig-like molecule, was shown to be highly expressed on chicken thymocytes and quickly down-regulated upon thymocyte exportation (peripheral expression half-life of 2-3 weeks). Current assessment of human and macaque thymic function is performed by evaluating TREC frequencies in total genomic DNA or total cell lysate from T cell populations, which both lead to cell death. Thus, studies aiming at discovering exclusive functional and phenotypic properties of RTEs are impossible until they can be accurately FACs-purified.

It would be highly desirable to be provided with the generation of a DNA construct model in which only TREC-containing T lymphocytes (e.g. RTEs) would express high levels of the green fluorescent protein (GFP), making them easily identifiable using conventional FACS technology. Thereby, mouse thymic function would be quantifiable by FACS analysis making the mouse of the present invention a perfect model for the screening of molecules potentially playing a role in enhancing/decreasing thymic exportation.

### SUMMARY OF THE INVENTION

In accordance with the present invention there is provided a DNA construct for in vivo expression in an excision DNA circle created by DNA recombination machinery in T cells from a non-human mammal which comprises two recombination signal sequences (RSS) consensus sequences flanking a promoter, an enhancer and a reporter gene, wherein said excision DNA circle is diluted out after cellular division and the excision DNA circle is detected by expression of the reporter gene and the detection is indicative of thymic function activity of the mammal.

The DNA construct in accordance with a preferred embodiment of the present invention for screening drugs enhancing and/or decreasing thymic function, wherein an increase of detection level being indicative of a drug enhancing thymic function and wherein a decrease of detection level being indicative of a drug decreasing thymic function, wherein the increase or decrease is compared to thymic function of the mammal prior to administration of drug.

The DNA construct in accordance with a preferred embodiment of the present invention, wherein the RSS consensus sequences are sequences recognized by proteins recombination activating genes (RAG)1 and RAG2.

The DNA construct of the present invention as set forth in FIG. 1.

In accordance with the present invention, there is provided a T cell transiently transfected with the DNA construct of the present invention, the cell expressing quantifiable levels of reporter gene for green fluorescent protein (GFP) for determining enhancing/decreasing thymic exportation.

The cell in accordance with a preferred embodiment of the present invention, wherein the DNA construct is introduced to the cell using a vector selected from the group consisting of: retroviral vector, recombinant vaccinia vector, recombinant pox virus vector, poliovirus, influenza virus, adenovirus, adeno-associated virus, herpes and HIV.

The cell in accordance with a preferred embodiment of the present invention, wherein the DNA construct is introduced to the cell using a physical method selected from the group consisting of: lipofection, direct DNA injection, microprojectile bombardment, electroporation, liposomes and DNA ligand.

In accordance with the present invention, there is provided a non-human mammal for in vivo screening molecules enhancing and/or decreasing thymic function in a subject, comprising a cell subtype from a non-human transfected with the DNA construct of the present invention, wherein the cell subtype after differentiation express quantifiable levels of reporter gene for determining enhancing/decreasing thymic exportation compared to thymic function prior administration of the molecules.

The mammal in accordance with a preferred embodiment of the present invention, wherein the cell is precursor of T lymphocyte.

The mammal in accordance with a preferred embodiment of the present invention, wherein the molecule is a potential modulator of thymic activity.

The mammal in accordance with a preferred embodiment of the present invention, wherein the mammal is selected from the group consisting of mouse, rat, chimpanzee and macaque.

In accordance with the present invention, there is provided a method for detecting recent thymic emigrant (RTE), the method comprising the steps of:
generating a transgenic mammal harboring the DNA construct of the present invention into its genome;
isolating lymphocytes from organ samples taken from the mammal;
analyzing the lymphocytes for detecting presence of cells expressing the reporter gene indicative of RTE.

In accordance with the present invention, there is provided a method for isolating RTE, the method comprising the steps of:
generating a transgenic mammal harboring the DNA construct of the present invention into its genome;
isolating lymphocytes from organ samples taken from the mammal;
analyzing the lymphocytes for detecting presence, of cells expressing the reporter gene indicative of RTE;
isolating the reporter gene expressing cells to obtain RTE.

The method in accordance with a preferred embodiment of the present invention, wherein the analyzing is performed by FACS analysis.

In accordance with the present invention, there is provided a method for in vivo quantification of thymopoiesis in a mammal, the method comprising the steps of:
generating a transgenic mammal harboring the DNA construct of the present invention into its genome;
isolating lymphocytes from organ samples taken from the mammal;
quantifying the amount of cells expressing the reporter gene from the lymphocytes
wherein the amount of cells expressing the reporter gene is indicative of thymopoiesis in a mammal.

The method in accordance with a preferred embodiment of the present application, wherein the quantifying is performed by FACS quantification.

In accordance with the present invention, there is provided a method for identifying a RTE phenotype, the method comprising the steps of:
generating a transgenic mammal harboring the DNA construct of the present invention into its genome;
isolating lymphocytes from organ samples taken from the mammal;
correlating expression of cytoplasmic and/or membrane bound molecule to a RTE phenotype.

The method in accordance with a preferred embodiment of the present invention, wherein the correlation is performed by FACS analysis and/or immunostrip assay.

In accordance with the present invention, there is provided a method for monitoring homeostasis of the RTE compartment in the mammal of the present invention, the method comprising the steps of:
generating a transgenic mammal harboring the DNA construct of the present invention into its genome;
correlating expression of cells of the mammal having his thymus ablated to a homeostasis of the RTE compartment in the mammal.

In accordance with the present invention, there is provided a method for monitoring homeostasis of the RTE compartment in the mammal of the present invention, the method comprising the steps of:
generating a transgenic mammal harboring the DNA construct of the present invention into its genome;
administering anti-human CD4 monoclonal antibodies to the mammal;
correlating expression of cells to a homeostasis of the RTE compartment in the mammal.

In accordance with the present invention, there is provided a method for monitoring homeostasis of the RTE compartment in the mammal of the present invention, the method comprising the steps of:
generating a transgenic mammal harboring the DNA construct of the present invention into its genome;
transferring reporter gene expressing cells of said mammal into syngenic recipient, said recipient having been thymectomized, irradiated or tolerized for said reporter gene.

In accordance with the present invention, there is provided a method for detection of extrathymic T cell production in a mammal, the method comprising the steps of:
generating a transgenic mammal harboring the DNA construct of the present invention into its genome;
eliminating thymic cells expressing the reporter gene in the mammal; and
correlating neo-synthesized reporter gene expressing cells with extrathymic T cell production in the mammal.

The method in accordance with a preferred embodiment of the present invention, wherein elimination of thymic cells expressing the reporter gene comprises thymectomy and administration of anti-human CD4 antibodies.

The method in accordance with a preferred embodiment of the present invention, wherein correlating neo-synthesized GFP+ cells comprises longitudinal FACS analysis.

For the purpose of the present invention the following terms are defined below.

The term “reporter gene” is intended to mean a GFP gene or any detectable gene that could be substituted, it may also have the same activity, it may also intend any fluorescent, radioactive label and any non fluorescent membrane-bound protein detected by a specific monoclonal or polyclonal antibody coupled to any label.

In the present invention, any strong promoter from viruses or eukaryotic cells could replace the promoter. Also, the enhancer could be replaced with any other strong enhancer. It is well known in the art what a strong promoter and a strong enhancer are and one skilled in the art will easily know what promoter and enhancer may be used to realize the present invention.

Also, even if the recombination signal sequences (RSS) disclosed in the present application are the preferred embodiment realized by the Applicant, RSS can still be “point-mutated” and replaced with some less efficient one and be functional. One skilled in the art would know how to proceed with such a mutation without affecting functionality of the RSS.

It is also understood that in order to target the recombination machinery where the transgene of the present application inserted itself, elements were incorporated ensuring that during TCRα rearrangement recombination of the transgene will occur. Exchanging those elements for TCRβ, TCRγ or TCRδ specific elements would definitively help tracking down other type of “newly produced” T cells (γδ if TCRγ or TCRδ elements for example).

In the present application, it is also understood that the mouse RTEs expressing GFP synthesize a truncate version of human CD4 molecule where the cytoplasmic domain of hCD4 is lacking, but that this gene could be replaced with any gene issued from any living organism without affecting the functionality of the present invention. This gene needs to have a transmembrane region and it could also be a soluble protein or peptide fused to a transmembrane region or a transmembrane protein.

All references herein are hereby incorporated by reference.

### BRIEF DESCRIPTION OF THE DRAWINGS

FIG. 1 illustrates the pre-rearrangement construct;

FIG. 2 illustrates the post-rearrangement construct;

FIG. 3 illustrates the efficiency of expression of the post-rearrangement constructs;

FIG. 4 illustrates a western blot analysis demonstrating that M12 and Dr3 cell lines express variable level of the Rag 2 protein, while the 1.8 cell line does not express Rag 2 protein; and

FIG. 5 illustrates flow cytometry assays performed 48 hours following the transfection of cell lines by pre-rearrangement constructs, where the GFP protein was measured.

### DETAILED DESCRIPTION OF THE INVENTION

In accordance with the present invention, there is provided a DNA construct for expression in an excision circle created by T and/or B cells DNA rearrangement, this circle being transitory and disappear after cellular division. This DNA construct is useful for screening drugs enhancing and/or decreasing thymic function.

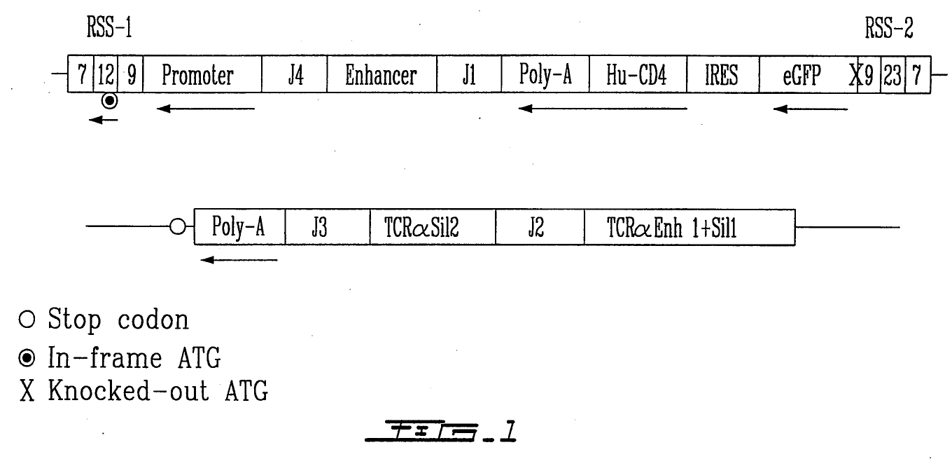

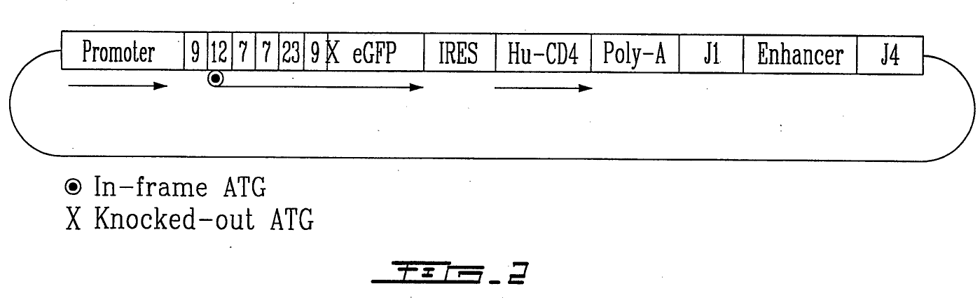

Given the fact that TREC are considered surrogate markers of thymic function irrespective of cell surface molecule expression, a DNA construct was engineered, this construct being used for introduction in a cell or a mammal as a dsDNA transgene. This transgene is bearing optimized recombination signal sequences (RSS) and TCRα locus-specific recombination elements that recruits the RAG machinery expressed during thymocyte ontogeny (FIG. 1). This rearrangement event generates a unique excision circle in which the viral SRα promoter is in-frame of the GFP gene, thereby making TREC-containing cells (e.g. newly produced T cells) GFP+.

A wide variety of methods may be utilized in order to deliver the DNA construct of the present invention to a warm-blooded animal or biological preparation. For example, within one embodiment of the invention, the vector construct is inserted into a retroviral vector, which may then be administered directly into a warm-blooded animal or biological preparation. Representative examples of suitable retroviral vectors and methods are described in more detail in the following U.S. patents and patent applications, all of which are incorporated by reference herein in their entirety: “DNA constructs for retrovirus packaging cell lines”, U.S. Pat. No. 4,871,719; “Recombinant Retroviruses with Amphotropic and Ectotropic Host Ranges”, PCT Publication No. WO 90/02806; and “Retroviral Packaging Cell Lines and Processes of Using Same”, PCT Publication No. WO 89/07150.

DNA construct may also be carried by a wide variety of other viral vectors, including for example, recombinant vaccinia vectors (U.S. Pat. Nos. 4,603,112 and 4,769,330), recombinant pox virus vectors (PCT Publication No. WO 89/01973), poliovirus (Evans et al. Nature, 339:385-388 (1989); and Sabin, J. Biol. Standardization, 1:115-118 (1973)); influenza virus (Luytjes et al., Cell, 59:1107-1113 (1989); McMichael et al., N. Eng. J. Med., 309:13-17 (1983); and Yap et al., Nature, 273:238-239 (1978)); adenovirus (Berkner, Biotechniques, 6:616-627 (1988); Rosenfeld et al., Science, 252:431-34 (1991)); adeno-associated virus (Samulski et al., J. Virol., 63:3822-3828 (1989); Mendelson et al., Virol., 166:154-165 (1988)); herpes (Kit, Avd. Exp. Med. Biol., 215:219-236 (1989)); and HIV (Poznansky, J. Virol., 65:532-536 (1991)).

In addition, DNA construct may be administered to warm-blooded animals or biological preparations utilizing a variety of methods, including, without limitation, lipofection (Felgner et al. Proc. Natl. Acad. Sci. USA, 84:7413-7417 (1989), direct DNA injection (Acsadi et al., Nature, 352:815-818 (1991)); microprojectile bombardment (Williams et al., PNAS, 88-2726-2730 (1991)); liposomes (Wang et al., PNAS, 84:7851-7855 (1987)); CaPO4 (Dubensky et al., PNAS, 81:7529-7533 (1984)); or DNA ligand (Wu et al., J. Biol. Chem., 264:16985-16987 (1989)).

Using the warm-blooded animal or biological preparation containing the DNA construct, it is possible to isolate real naïve cell irrespective of cell surface molecule expression patterns. A fraction of RTE is detected (those that have not yet loss their TREC due to the dilution effect).

It is well documented that B cells also express the same recombination machinery at some point in their development. This could lead to in vivo transgene rearrangement in B cells, making them fluorescent and generate “false positive”. In order to circumvent that, TCRα locus-specific elements are inserted downstream of the transgene (Winoto A, Baltimore D. EMBO J. 1989 March;8(3):729-33. Winoto, A. and Baltimore, D. Cell 59 (4), 649-655 (1989)), ensuring the cell type specificity of the rearrangement. The function of these elements is to ensure that the transgene of the present invention becomes accessible to the RAG machinery during T cell ontogeny.

In order to ensure a tight regulation of GFP fluorescence emission despite the integration site in the host chromosome, stop codons are inserted 3′ of the GFP gene (between the GFP and TCRα locus-specific elements). Furthermore, the endogenous ATG start codon from the GFP gene is “knocked-out” and inserted within the 23 bp spacer located within one RSS (FIG. 1). Given this, not leak in GFP fluorescence from cells that do not express the RAG machinery at one point in their life is anticipated.

Also, in order to study the in vivo replenishment rate of RTE, an IRES-hCD4 fragment is inserted in the transgene. Given the fact that this fragment is located on the excised DNA circle, downstream from the GFP but upstream from the polyadenylation site, this hCD4 protein will be produced together with GFP. Thus, the injection of an antibody directed against hCD4 would deplete (via the complement pathway) all GFP+ T cells (e.g. RTEs). This fragment may be viewed as a “reset button” for RTEs production.

These are the elements generated using the PCR technology and standard molecular biology techniques.
Backbone cloning vector: Stratagene pBSKS (Bluescript)
GFP gene: Clontech peGFP-C1
TCRα enhancer 1 and silencer 1: mouse genomic DNA
Silencer 2: mouse genomic DNA
SRα0 viral promoter (Takebe et al, 1988. Mol Cell Biol 8466: 72)
Junk 1, 2 and 3: human genomic DNA
CD3 enhancer: mouse genomic DNA
IgM enhancer: mouse genomic DNA
CD3δ promoter: mouse genomic DNA
IRES-hCD4: cloned IRES and human truncated CD4 (no cytoplasmic tail)
   “Proof-of-Concept” Validation

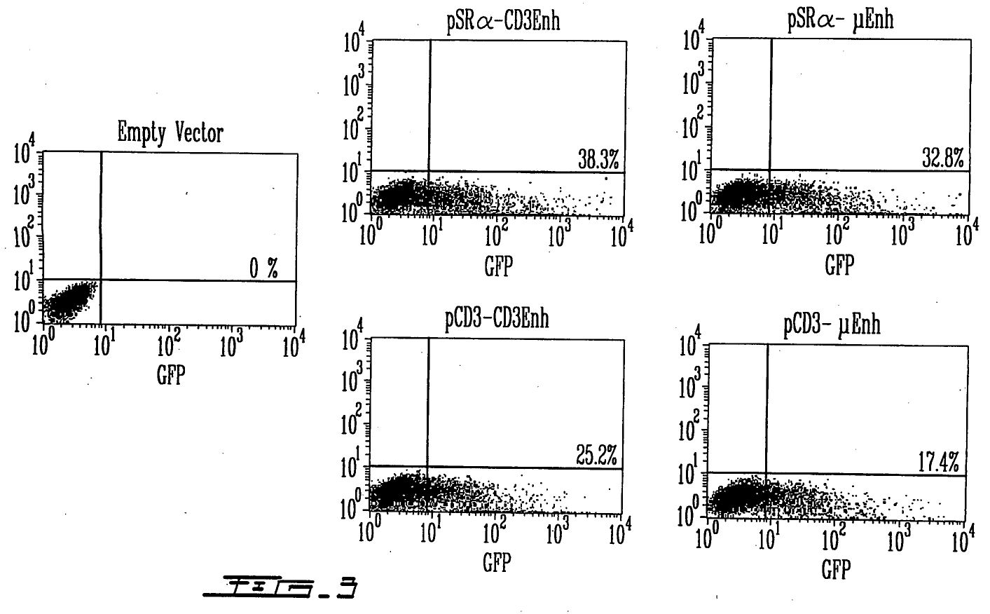

dsDNA constructs that exactly simulate the end product of the rearrangement events (see FIG. 2) were designed and generated. These constructs are referred to as the “post-rearrangement” constructs. The various elements constituting these constructs were sequentially introduced in pBSKS (Bluescript) cloning vector, in which we had previously exchanged the multiple cloning site (MCS) region with one bearing the required restriction sites. This allowed us to optimize the promoter/enhancer combination that will be used in the final constructs (Table 1).

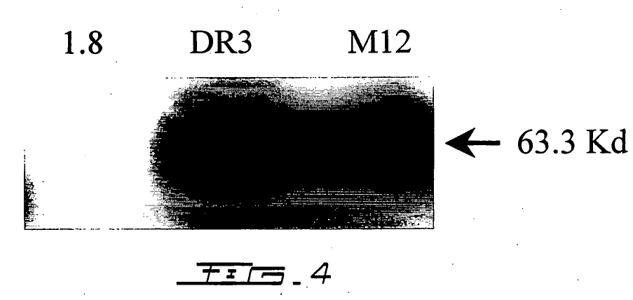

An example of GFP expression following transient transfection of 50 μg of the “post-rearrangement constructs” in 5×10⁶ Jurkat E6.1 cell line is shown in FIG. 3. As shown in Table 1, variable GFP expression was observed using the different constructs. Transfection experiments performed in Jurkat E6.1 cell line were able to demonstrate that the Srα promoter, coupled to the CD3δ enhancer is the best combination to express GFP (underlined data) in the post-rearrangement constructs in this particular T cell line (table 1a). As shown in table 1b, the efficiency of this combination was confirmed in DR3 cells that constitutively express the Rag1 and Rag2 proteins, and consequently was used in the next series of experiments.

TABLE 1
Optimization of promoter/enhancer combination
a-Experiment 1

| Jurkat-E6.1 | % of GFP+ Cells | Mean Fluorescence |
|---|---|---|
| pBSKS-MCS-JFR | 0.07 | 7.48 |
| peGFP-C1 | 64.04 | 1260.19 |
| pSRα-CD3enh | 35.44 | 914.69 |
| pSRα-CD3enh | 38.15 | 540.32 |
| pSRα-CD3enh | 31.84 | 341.84 |
| pSRα-μenh | 26.19 | 276.92 |
| pSRα-μenh | 31.71 | 407.05 |
| pCD3δ CD3enh | 24.76 | 214.18 |
| pCD3δ CD3enh | 24.54 | 163.17 |
| pCD3δ-μenh | 26.62 | 226.85 |

b-Experiment 2

| DR3 | % of GFP+ Cells | Mean Fluorescence |
|---|---|---|
| no DNA | 0.09 | 10.34 |
| peGFP-C1 | 96.32 | 2599.87 |
| pSRα-CD3enh | 87.98 | 482.35 |
| pSRα-CD3enh | 81.85 | 408.39 |
| pCD3δ-CD3enh | 32.50 | 183.53 |
| pCD3δ-μenh | 62.69 | 452.55 |

Experiment 3

| DR3 | % of GFP+ Cells | Mean Fluorescence |
|---|---|---|
| pBSKS-MCS-JFR | 0.08 | 10.75 |
| peGFP-C1 | 97.35 | 3458.11 |
| pCD3δ-CD3enh | 39.42 | 389.67 |
| pCD3δ-μenh | 63.87 | 636.87 |
| pSRα-CD3enh | 97.94 | 1510.26 |

Legend to Table 1:
Transfection experiments of the post rearrangement constructs.
a) Four different DNA constructs were transfected into Jurkat-E6.1 cell line. GFP expression was observed for all construct, the pSRα-CD3enh combination being the more efficient (underlined data).
b) The same constructs were transfected into Rag1/2 expressing DR3 cell line. Again, the pSRα-CD3enh construct leads to higher GFP expression levels (underlined data). For these experiments, pBSKS-MCS-JFR is a negative control and peGFP-C1 is a positive control.

Once the optimal elements were identified (the SRα promoter and the CD3δ enhancer), the non-rearranged dsDNA transgene (e.g. the “pre-rearrangement” construct) was synthesized and tested for its ability to recombine in vitro using RAG-1/2 expressing cell lines.

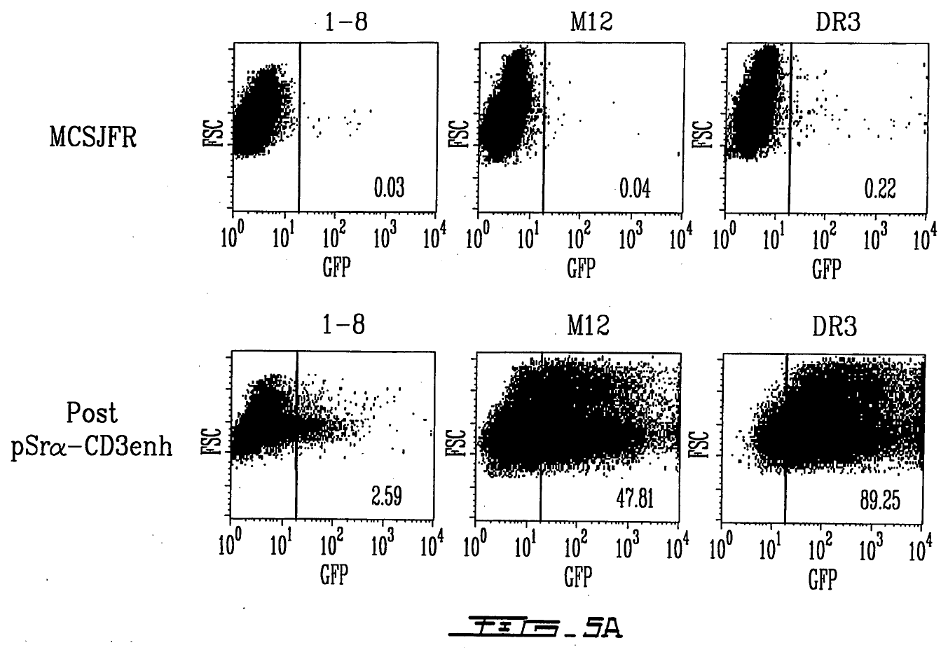

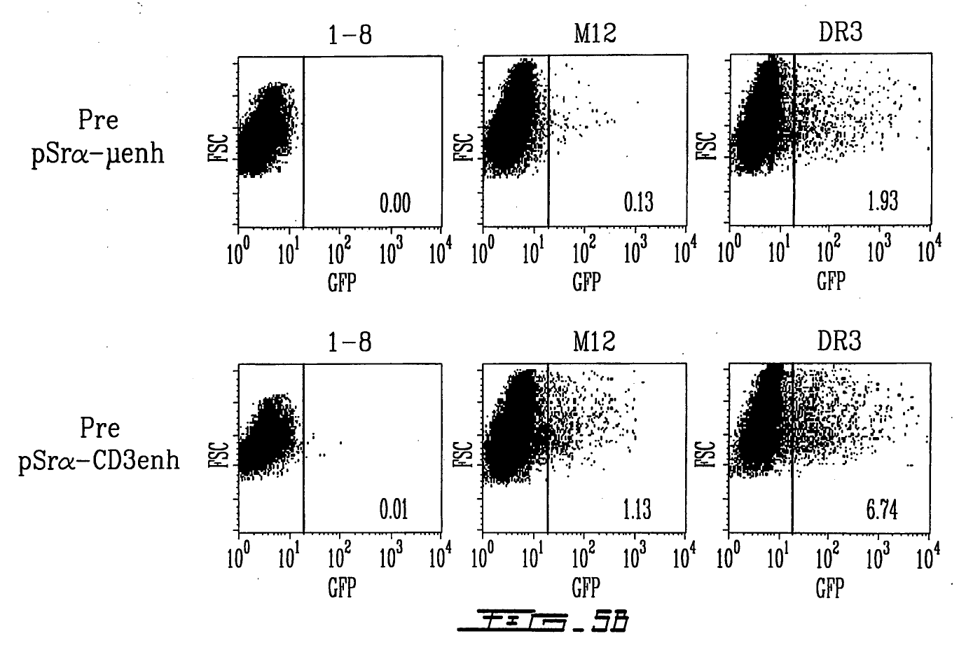

Several constructs were generated with the CD3δ or the SRα promoter With either the CD3δ or the μEnhancer. These DNA constructs were transfected into the 1-8, M12 and Dr3 cell lines that express variable level of the Rag 2 protein, as demonstrated by western blot analysis (FIG. 4). In these cell lines, the expression of the GFP protein was measured by flow cytometry analysis 48 hours following transfection (FIG. 5). In 3 independent experiments (Table 2), GFP was detected in all the Rag expressing cell lines (i.e. M12 and DR3) while the 1.8 cell line remained negative for the expression of GFP (Table 2a). Of note is the fact that the 1.8 cell line is able to express GFP when transfected with the post-rearrangement constructs (Table 2a), demonstrating that the lack of expression is a consequence of the inability of these cells to rearrange the pre-rearrangement construct due to the absence of Rag expression.

These results were confirmed in several independant experiments (Table 2b). Experiment 2 demonstrates that the expression of the GFP following transfection in the Rag-expressing cell line is due to rearrangement of the DNA construct and not to non-specific GFP transcription through an unknown promoter located 3′ of the GFP gene. This is demonstrated by GFP expression in transfection experiments with Not I and Bgl II digested DNA constructs. These restriction enzymes are able to digest the pre-rearrangement construct 5′ of the 5′-RSS and 3′ of the 3′-RSS, thus removing parts of the construct susceptible to contain non-specific recombination sequences or cryptic promoters.—

TABLE 2
In vitro Transfection experiments
a-Experiment # 1

| | | | Mean Fluo. of |
|---|---|---|---|
| Cells | Plasmids | % of GFP+ Cells | GFP+ cells |
| 1-8 | pBSKS-MCS-JFR | 0.03 | 100.88 |
| M12 | pBSKS-MCS-JFR | 0.04 | 102.96 |
| DR3 | pBSKS-MCS-JFR | 0.22 | 695.07 |
| 1-8 | Post-pSRα-CD3enh | 2.59 | 27.17 |
| M12 | Post-pSRα-CD3enh | 47.81 | 211.43 |
| DR3 | Post-pSRα-CD3enh | 89.25 | 756.52 |
| 1-8 | Pre-pSRα-CD3enh | 0.01 | 17.29 |
| M12 | Pre-pSRα-CD3enh | 1.13 | 49.31 |
| DR3 | Pre-pSRα-CD3enh | 6.74 | 153.79 |

b-Experiment # 2

| | | | Mean Fluo. of |
|---|---|---|---|
| Cells | Plasmids | % of GFP+ Cells | GFP+ cells |
| M12 | pBSKS-MCS-JFR | 0.46 | NA |
| DR3 | pBSKS-MCS-JFR | 0.07 | NA |
| M12 | Pre-pSRα-CD3enh | 1.41 | 42.04 |
| DR3 | Pre-pSRα-CD3enh | 8.32 | 123.02 |
| M12 | Pre-SRα-CD3enh | 2.56 | 42.22 |
| | (Not 1-Bgl II) | | |
| DR3 | Pre-SRα-CD3enh | 15.01 | 79.54 |
| | (Not 1-Bgl II) | | |
| Legend to table 2: | | | |
| Transfection experiments of the Pre-rearrangement construct. | | | |
| a) The Pre rearrangement construct (pSRα-CD3enh) was transfected into several cell lines, expressing variable levels of Rag1/2 proteins. | | | |
| # GFP expression correlates to Rag expression. The positive control (pSRα-CD3enh - post rearrangement construct) is expressed in all tested cell lines. | | | |
| b) The expression of GFP is not due to non-specific transcription though an unknown promoter located 3′ of the GFP gene in the pre-rearrangement construct. | | | |
| # DNA construct was digested in order to excise the sequence susceptible to be rearranged, following transfection, these digested constructs are able to express GFP showing that this expression is a consequence of DNA rearrangement. | | | |
| # In these experiments the PBSKS-MCS-JFR plasmid was used as a negative control while the post rearrangement construct Post-pSrαCD3enh served as a positive control. | | | |

### EXAMPLE I

### Extensive Phenotypic Characterization of Mouse Recent Thymic Emigrants

In order to proceed with the identification of a mouse RTE-specific phenotype, GFP^{high} PBMC isolated from the mice is phenotypically characterized using a multiple mouse monoclonal antibodies directed against CD4, CD3, CD8, TLA4, CD28, CD95, CD27, ICAM-1, α₄β₇ integrin, chemokine and hormone receptors (GM-CSF, c-kit).

### EXAMPLE II

### Mice Crosses with Mouse Cytokine/Chemokine Knock-Out

As a novel way to determine the role of any cytokine (in this case IL-7) on thymopoiesis regulation, mice are crossed with the IL-7 knock-out mice given the fact that IL-7 plays an important role in the maintenance/survival of the naïve T cell compartment. The end-product of this crossing is a cytokine or chemokine knock-out mice in which RTE can be detected, quantified and isolated.

### EXAMPLE III

### Extrathymic Generation of T Cells

Hematopoietic stem cells (T cells precursors c-Kit⁺, Ly-6A/E⁺, Lin⁻) isolated from day 14 fetal liver of a mouse is infused in thymectomized or sham-thymectomized irradiated syngenic and congenic mice. Longitudinal studies measuring the rate of appearance of GFP⁺ T cells is done on both groups. If present, the identification of the organ responsible for de novo extrathymic production of T cells (gut-associated lymphoid tissue (GALT), spleen or possibly lymph nodes) will be identifiable by fluorescence detection.

### EXAMPLE IV

### Determination of RTE Cell Activation Requirements

Recent thymic emigrants may need to undergo maturation steps before becoming real functional naïve T cells able to respond to antigens. This is fully compatible with recent experiments demonstrating that naïve T cells can “homeostatically” proliferate without loosing their naïve phenotype. It is possible that these rounds of replication remodel the chromatin, making some transcriptionaly-inactive genes expressed (Kieper W C, Jameson S C. Proc Natl Acad Sci USA. 1999 Nov. 9;96(23):13306-11. Goldrath A W, Bogatzki L Y, Bevan M J. J Exp Med. 2000 Aug. 21;192(4):557-64.) To answer that, FACS-purified GFP^{High} T cells (e.g. “real” recent thymic emigrants) are stained with CFSE, a cell division marker. RTE stimulation is done using anti-CD3 and anti-CD28 antibodies and cytokines production monitored by FACS analysis. With this, the number of rounds of replication required for RTE to reach functional maturity can be determined.

### EXAMPLE V

### Compartmentalization of RTE

Where do new cells go when they are produced? It is though that naïve T cells go into lymph nodes (where potential antigens are likely to be presented) once they are generated. In our model, tracking-down RTE can be done using histological slides of various peripheral organs (lymph nodes, spleen, gut-associated lymphoid tissue). Infusion into normal mice of hematoipoietic stem cells previously isolated from the mouse followed by histological studies help understanding the faith of de novo produced T cells.

### EXAMPLE VI

### Do RTE Contribute to the Maintenance/Re-Seeding of the SIV Reservoir?

Ex vivo transfection of Macaca Mulata CD34+ precursors cells with our transgene followed by re-infusion in irradiated SIV-infected hosts bearing or not a thymus help investigating if RTE harbor SIV proviral DNA, thereby assessing the contribution of ongoing thymopoiesis to the SIV reservoir. Thymocytes were shown to be infected both in vivo and in vitro. Again, GFP^{High} T cells are FACS-purified and SIV proviral DNA quantified using the LightCycler™ real-time on-line quantitative PCR technology available now in the lab. Of course, the mouse specific elements have to be replaced by their homologue in the macaque model.

Also, analogous experiments previously performed in mice can be done in the macaque model using the same “pre-rearrangement” transgene.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

1. A DNA construct for in vivo expression in an excision DNA circle created by DNA recombination machinery in T cells from a non-human mammal which comprises two recombination signal sequences (RSS) consensus sequences flanking a promoter, an enhancer and a reporter gene, wherein said excision DNA circle is diluted out after cellular division and said excision DNA circle is detected by expression of said reporter gene and said detection is indicative of thymic function activity of said mammal.

2. The DNA construct of claim 1 for screening drugs enhancing and/or decreasing thymic function, wherein an increase of detection level being indicative of a drug enhancing thymic function and wherein a decrease of detection level being indicative of a drug decreasing thymic function, wherein said increase or decrease is compared to thymic function of said mammal prior to administration of drug.

3. The DNA construct of claim 1, wherein said RSS consensus sequences are sequences recognized by proteins recombination activating genes (RAG)1 and RAG2.

4. A DNA construct of claim 1 as set forth in FIG. 1.

5. A T cell transiently transfected with the DNA construct of claim 1, said cell expressing quantifiable levels of reporter gene for green fluorescent protein (GFP) for determining enhancing/decreasing thymic exportation.

6. The cell of claim 5, wherein said DNA construct is introduced to said cell using a vector selected from the group consisting of: retroviral vector, recombinant vaccinia vector, recombinant pox virus vector, poliovirus, influenza virus, adenovirus, adeno-associated virus, herpes and HIV.

7. The cell of claim 5, wherein said DNA construct is introduced to said cell using a physical method selected from the group consisting of: lipofection, direct DNA injection, microprojectile bombardment, electroporation, liposomes and DNA ligand.

8. The cell of any one of claim 5, wherein said RSS consensus sequences are sequences recognized by proteins RAG1 and RAG2.

9. The cell of any one of claim 5, wherein said DNA construct is a DNA construct as set forth in FIG. 1.

10. A non-human mammal for in vivo screening molecules enhancing and/or decreasing thymic function in a subject, comprising a cell subtype from a non-human transfected with the DNA construct of claim 1, wherein said cell subtype after differentiation express quantifiable levels of reporter gene for determining enhancing/decreasing thymic exportation compared to thymic function prior administration of said molecules.

11. The mammal of claim 10, wherein said cell is precursor of T lymphocyte.

12. The mammal of claim 10, wherein said molecule is a potential modulator of thymic activity.

13. The mammal of claim 10, wherein said mammal is selected from the group consisting of mouse, rat, chimpanzee and macaque.

14. The mammal of claim 10, wherein said mammal is a mouse.

15. The mammal of claim 10, wherein said RSS consensus sequences are sequences recognized by proteins RAG1 and RAG2.

16. The mammal of claim 10, wherein said DNA construct is a DNA construct as set forth in FIG. 1.

17. A method for detecting recent thymic emigrant (RTE), said method comprising the steps of:—generating a transgenic mammal harboring the DNA construct of claim 1 into its genome;—isolating lymphocytes from organ samples taken from said mammal;—analyzing said lymphocytes for detecting presence of cells expressing said reporter gene indicative of RTE.

18. A method for isolating RTE, said method comprising the steps of:—generating a transgenic mammal harboring the DNA construct of claim 1 into its genome;—isolating lymphocytes from organ samples taken from said mammal;—analyzing said lymphocytes for detecting presence of cells expressing said reporter gene indicative of RTE;—isolating said reporter gene expressing cells to obtain RTE.

19. The method of claim 17, wherein said analyzing is performed by FACS analysis.

20. The method of claim 17, wherein said mammal is selected from the group consisting of mouse, rat, chimpanzee and macaque.

21. The method of claim 17, wherein said mammal is a mouse.

22. The method of claim 17, wherein said RSS consensus sequences are sequences recognized by proteins RAG1 and RAG2.

23. The method of claim 17, wherein said DNA construct is a DNA construct as set forth in FIG. 1.

24. A method for in vivo quantification of thymopoiesis in a mammal, said method comprising the steps of:—generating a transgenic mammal harboring the DNA construct of claim 1 into its genome;—isolating lymphocytes from organ samples taken from said mammal;—quantifying the amount of cells expressing said reporter gene from said lymphocytes wherein said amount of cells expressing said reporter gene is indicative of thymopoiesis in a mammal.

25. The method of claim 24, wherein said quantifying is performed by FACS quantification.

26. The method of claim 24, wherein said mammal is selected from the group consisting of mouse, rat, chimpanzee and macaque.

27. The method of claim 24, wherein said mammal is a mouse.

28. A method for identifying a RTE phenotype, said method comprising the steps of:—a transgenic mammal harboring the DNA construct of claim 1 into its genome;—isolating lymphocytes from organ samples taken from said mammal);—correlating expression of cytoplasmic and/or membrane bound molecule to a RTE phenotype.

29. The method of claim 28, wherein said correlating is performed by FACS analysis and/or immunostrip assay.

30. The method of claim 28, wherein said phenotype is the phenotype of a mammal selected from the group consisting of mouse, rat, chimpanzee and macaque.

31. The method of claim 28, wherein said mammal is a mouse.

32. A method for monitoring homeostasis of the RTE compartment in the mammal of claim 10, said method comprising the steps of:—generating a transgenic mammal harboring the DNA construct of claim 1 into its genome;—correlating expression of cells of said mammal having his thymus ablated to a homeostasis of the RTE compartment in said mammal.

33. A method for monitoring homeostasis of the RTE compartment in the mammal of claim 10, said method comprising the steps of:—generating a transgenic mammal harboring the DNA construct of claim 1 into its genome;—administering anti-human CD4 monoclonal antibodies to said mammal; correlating expression of cells to a homeostasis of the RTE compartment in said mammal.

34. A method for monitoring homeostasis of the RTE compartment in the mammal of claim 10, said method comprising the steps of:—generating a transgenic mammal harboring the DNA construct of claim 1 into its genome;—transferring reporter gene expressing cells of said mammal into syngenic recipient, said recipient having been thymectomized, irradiated or tolerized for said reporter gene.

35. The method of claim 32, wherein said subject is selected from the group consisting of mouse and macaque.

36. The method of claim 32, wherein said subject is a mouse.

37. The method of claim 32, wherein said RSS consensus sequences are sequences recognized by proteins RAG1 and RAG2.

38. The method of claim 32, wherein said DNA construct is a DNA construct as set forth in FIG. 1.

39. A method for detection of extrathymic T cell production in a mammal, said method comprising the steps of:—generating a transgenic mammal harboring the DNA construct of claim 1 into its genome;—eliminating thymic cells expressing said reporter gene in said mammal; and—correlating neo-synthesized reporter gene expressing cells with extrathymic T cell production in said mammal.

40. The method of claim 39, wherein elimination of thymic cells expressing said reporter gene comprises thymectomy and administration of anti-human CD4 antibodies.

41. The method of claim 39, wherein correlating neo-synthesized GFP+ cells comprises longitudinal FACS analysis.

42. The method of claim 39, wherein said mammal is selected from the group consisting of mouse and macaque.

43. The method of claim 39, wherein said mammal is a mouse.

44. The method of claim 18, wherein said analyzing is performed by FACS analysis.

45. The method of claim 18, wherein said mammal is selected from the group consisting of mouse, rat, chimpanzee and macaque.

46. The method of claim 18, wherein said mammal is a mouse.

47. The method as claimed in claim 18, wherein said RSS consensus sequences are sequences recognized by proteins RAG1 and RAG2.

48. The method as claimed in claim 18, wherein said DNA construct is a DNA construct as set forth in FIG. 1.

49. The method of claim 33, wherein said subject is selected from the group consisting of mouse and macaque.

50. The method of claim 34, wherein said subject is selected from the group consisting of mouse and macaque.

51. The method of claim 33, wherein said subject is a mouse.

52. The method of claim 34, wherein said subject is a mouse.

53. The method of claim 33, wherein said RSS consensus sequences are sequences recognized by proteins RAG1 and RAG2.

54. The method of claim 34, wherein said RSS consensus sequences are sequences recognized by proteins RAG1 and RAG2.

55. The method of claim 33, wherein said DNA construct is a DNA construct as set forth in FIG. 1.

56. The method of claim 34, wherein said DNA construct is a DNA construct as set forth in FIG. 1.
